(19) United States
(12) Patent Application Publication
(10) Pub. No.: US 2011/0092993 A1
(43) Pub. Date: Apr. 21, 2011

(54) TISSUE APPROXIMATING DEVICE

(76) Inventor: MOISES JACOBS, Miami, FL (US)

(21) Appl. No.: 12/908,070

(22) Filed: Oct. 20, 2010

Related U.S. Application Data
(60) Provisional application No. 61/253,182, filed on Oct. 20, 2009.

(51) Int. Cl. A61B 17/11

(52) U.S. Cl. 606/153

(57) ABSTRACT

The present invention is referred to a tissue approximating device comprising a two-piece anastomosis clamp that can be used to approximate two tissue sections together via an open or laparoscopic technique, via Natural Orifice Translumenal Endoscopic Surgery (NOTES) or via single site surgery. The clamp may be releasable attached to a clamp applying instrument for delivery in accordance with such procedures. The clamp includes a first member and a second member, where the clamp members are operable configured to fasten together to clamp and hold tissue, in juxtaposition to establish an anastomosis. The first clamp member includes a set of spikes and the second clamp member includes a set of receptacles for receiving said spikes. Said members may have a rectilinear shape depending on the type of tissue to be anastomosed, and they are made of an absorbable material. During the surgical procedure, a soft curtain-like covering made also of an absorbable or biodegradable material will be placed over said first and second members before the procedure as well as over the tissue to be anastomosed.

This application claims the benefit of co-pending U.S. Provisional Patent Application Ser. No. 61/253,182, filed on Oct. 20, 2009, which is incorporated herein in its entirety.

### BACKGROUND OF THE INVENTION

1. Field of the Invention

The present invention relates, in general, to surgical devices and more particularly is referred to a visceral tissue approximating device comprising a two-piece anastomosis clamp that can be used to approximate two tissue sections together via an open or laparoscopic technique, via Natural Orifice Translumenal Endoscopic Surgery (NOTES) or via single site surgery. Even more particularly, the present invention is referred to a visceral tissue approximating method and device, especially useful for approximating and anastomosing visceral tissue such as, for example, bowel tissue.

2. Description of the Prior Art

Many surgical procedures involve a suturing maneuver. Suturing of body tissues is a time consuming aspect of most surgical procedures. Modern surgical procedures tend to avoid performing a large opening to expose the area. Instead, performing small incisions to introduce endoscopes or laparoscopes is preferred in conjunction with specialized surgical instrumentation to detect, diagnose, and repair areas.

Laparoscopic surgery, also called minimally invasive surgery (MIS), is a modern surgical technique in which operations are performed through small incisions (usually 0.5-1.5 cm) as compared to larger incisions needed in traditional surgical procedures.

There are a number of advantages to the patient with laparoscopic surgery versus an open procedure. These include:
less post operative discomfort since the incisions are much smaller;
quicker recovery times;
shorter hospital stays;
earlier return to full activities;
much smaller scars;
less internal scarring;
reduced pain from infection and hemorrhaging, between others.

Access to the operative site using minimally invasive techniques is accomplished by inserting small tubes called ports into a body cavity. These tubes have a diameter of, for example, half an inch. There are several instruments in the prior art for suturing within a body cavity through these port tubes like the one described by Mulhollan et al. in the U.S. Pat. No. 4,621,640.

Some common examples of laparoscopic surgeries are: gallbladder removal, hernia repair, appendix removal, tubal ligation, etc. Depending on the type of surgery to be performed is the part of the body in which the incision may be made.

Natural orifice translumenal endoscopic surgery (NOTES) is a surgical technique whereby scarless abdominal operations can be performed with an endoscope passed through a natural orifice (mouth, urethra, anus, etc.) then through an internal incision in the stomach, vagina, bladder or colon, thus avoiding any external incisions or scars. Natural orifice translumenal endoscopic surgery generally requires no incisions because instruments, such as staple guns, can be inserted through the mouth and snaked down the esophagus. If the work to be done involves, for example, the lower portion of the intestines, the instruments can be inserted through the rectum. Another procedure known as transvaginal approach for the placement of the sutures has found favor among physicians. F instance, a nephrectomy may be performed with a transvaginal retroperitoneal NOTES technique. Nephrectomy performed through a natural orifice could minimize postoperative recovery. The vagina has been considered a viable route for kidney retrieval also following laparoscopic nephrectomies.

Hence, alternatives have been sought to use minimally invasive surgery, which would include laparoscopic surgery, or the new approach called “mini-incision surgery” using the principles of traditional open surgery, along with some of the equipment advances of laparoscopy.

In all the above cited procedures, an anastomosis is involved. Anastomosis is a surgical connection between two structures. It usually means a connection that is created between tubular structures, such as blood vessels or loops of the intestine. For example, when part of an intestine is surgically removed, the two remaining ends are sewn or stapled together (anastomosed), and the procedure is referred to as an intestinal anastomosis. There are several surgical procedures in which are necessary to perform an anastomosis, and even though there are several different types of devices and techniques for doing that, a clamp is one of them.

There are particular cases of anastomoses that pose special threads or risks to the patient like the anastomoses of the bowel. One of the risky situations in which the life of the patient is in risk relates to the possibility of the leakage of internal fluids into the abdominal cavity. In accordance with different statistics, between 3-5% of the bowel anastomosis procedures presents a leakage, and every leakage means the life of the patient is in great risk.

For example, in cases in which a section of the bowel must be removed, once the cut-off procedure is completed, the loose ends of the bowel must be re-connected or linked. This is a very well known procedure usually involving clipping the ends of each portion of the bowel. The problem resides on the fact that the gastric fluids start flowing through the wound and if any deficiency in the anastomosis is found, said fluids may go through the wound to the abdominal cavity, creating a life-threatening situation for the patient.

There are several tissue approximating devices and surgical staplers for performing the anastomosis in the prior art. For example, US Patent Application Serial N° 20080114383 filed by Hunt et al. provides a two-piece anastomosis fastener that can be used to join two tissue sections together in accordance with Natural Orifice Transendoscopic Surgery (NOTES). The fastener may be releasably attached to a fastener applying instrument for delivery in accordance with such procedures. The fastener includes a first member and a second member, where the clamp members are operably configured to fasten together to clamp and hold tissue, such as gastric tissue, in juxtaposition to establish an anastomosis. The first clamp member and the second clamp member are coupled with an adhesive.

U.S. Pat. No. 7,033,370 granted to Gordon et al. teaches about a method and device for the placement of sutures and for the purpose of approximating tissue. The invention relates to devices for approximation, ligation and fixation of tissue using a suture, to various constituent parts comprising said devices, and particularly to the placement of sutures into certain difficult to access ligamental structures, to the approximation of tissue separated by means of an endosurgical trocar being inserted into a body cavity, and to approximation, ligation, and fixation of body tissue using both traditional open surgical and endosurgical techniques and instruments. Loading of suture material including needles into the device is also included, and the introduction and placement of the device into the body cavity, with the distal end having deployable needle guides, extending the needle guides either simultaneously or individually to the periphery of the wound, engaging the wound with the needle guides, driving the needles and suture material through the tissue to be approximated into a catch mechanism, retracting the needle guides and withdrawing the device, leaving a loop of suture material in the margin of tissue. The suture may then be tied to approximate the wound and excess suture material cut off.

U.S. Pat. No. 6,997,932 granted to Dreyfuss et al. shows a device for the placement of sutures and for the purpose of approximating tissue includes an elongate body member, a suture deployment system, and a catch. Methods of placing sutures include inserting and deploying in a patient a device including an elongate body member, a suture deployment system, and a catch are also encompassed. The suture deployment system is disposed at a distal portion of the elongate body member, and includes a suture carrier having a sharpened distal end for tissue penetration and a notch for holding a formed suture tip. The catch is disposed on the elongate body member to receive and retain the formed suture tip.

Also U.S. Pat. No. 5,735,445 granted to Vidal et al. teaches about a surgical stapler having a supporting frame, replaceable staple cartridge, an anvil, a mechanism for approximating the cartridge relative to the anvil, and a mechanism for firing the device so as to crimp the staples against the anvil in a manner to enable the surgeon to substantially simultaneously place one or more rows of surgical staples in organs or tissue. The device, while at all times permitting approximation of the cartridge relative to the anvil, provides a lockout feature for preventing retiring if the staple cartridge is spent. In this way, the device can be used as a clamping mechanism even after the staples have been fired.

U.S. Pat. No. 5,678,748 granted to Plyley et al. is referred to a surgical stapler for use by a surgeon to place one or more rows of surgical staples in organs or tissue. The stapler is of a character having a supporting frame, a replaceable staple cartridge, an anvil, a mechanism for approximating the cartridge relative to the anvil, and a mechanism for firing the device so as to crimp the staples against the anvil. The device, while at all times permitting approximation of the cartridge relative to the anvil, provides a novel safety mechanism that clearly indicates to the surgeon that the staples have been fired from the staple cartridge and simultaneously disables the device until it is manually reset.

Finally, U.S. Pat. No. 6,926,724 describes temporary biocompatible stent and method for visceral anastomosis. The stent is provided with integral means for maintaining the structural stability of the stent while providing substantial flexibility. The method comprises fitting the luminal stumps of the viscus over either end of a stent of the invention, and joining the ends of each stump together. A short time after completion of the anastomosis, the stent dissolves and is absorbed safely into the body. The stent and method can be beneficially used in laparoscopic or more invasive traditional surgical procedures. The stent and method are particularly well suited for anastomosis of the bowel.

None of the above described devices or methods involves a safe and practical device and method through which the bowel anastomosis procedure may be performed providing to the surgeon and the patient the peace of mind that no leakage of gastric fluids to the abdominal cavity will occur. As such, a tissue approximating method and device for performing the anastomosis of visceral tissue like for example the bowel through a fast and simple procedure is still desired in the market.

### SUMMARY OF THE INVENTION

A main object of the present invention is to provide a tissue approximating device comprising a two-piece anastomosis clamp that can be used to approximate two tissue sections together via an open or laparascopic technique, via Natural Orifice Transendoscopic Surgery (NOTES) or via single site surgery.

It is another object of the present invention to provide a clamp device for performing the anastomosis comprising a two-piece rectilinear anastomosis clamp, the first piece including a set of spikes and the second piece including a set of complementary orifices in which said spikes can be fitted in.

Yet another object of the present invention is to provide a tissue approximating device made of absorbable or biodegradable material. This device can be absorbed by the body several weeks or months after the procedure without the necessity of performing a new surgical procedure to take it out of the patient.

Another object of the present invention is to provide a biodegradable and absorbable approximating device capable of being absorbed by the patient's body several weeks after the procedure without the necessity of performing a new surgical procedure for taking the device out of the patient's body.

In summary, the present invention is referred to a tissue approximating device comprising a two-piece anastomosis clamp that can be used to approximate two tissue sections together via an open technique, via Natural Orifice Translumenal Endoscopic Surgery (NOTES) or via single site surgery. The clamp may be releasable attached to a clamp applying instrument for delivery in accordance with such procedures. The clamp includes a first member and a second member, where the clamp members are operable configured to fasten together to clamp and hold tissue, in juxtaposition to establish an anastomosis. The first clamp member includes a set of spikes, and the second clamp member includes a set of receptacles for receiving said spikes. Said members may have a rectilinear or curved shape depending on the type of tissue to be anastomosed, and they are made out of an absorbable material. During the surgical procedure, a soft curtain-like covering made also of an absorbable or biodegradable material will be placed over said first and second members before the procedure as well as over the tissue to be anastomosed. In addition, this biodegradable or absorbable curtainous material may be incorporated or be part of the first member with the spikes and the second member with the receptacles. Furthermore, the first and second member may have a lit through which this curtaineous material can be passed and accommodated about the tissues before firing (closing) the clamp.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 is an exploded perspective view of a preferred embodiment of the tissue approximating device of the present invention.

FIG. 2 is a cross-sectional view of the clamp device of the present invention of FIG. 1 taken along line A-A showing the first clamp member and the second clamp member prior to engagement.

FIG. 3 is a cross-sectional view of the purposed clamp device of FIG. 1 shown attached to a fold of tissue sandwiched between the first member and the second member.

FIG. 4 is another general perspective view of the purposed tissue approximating device shown attached to a fold of tissue, for example, a gastric or vaginal tissue.

### DETAILED DESCRIPTION OF REPRESENTATIVE EMBODIMENTS

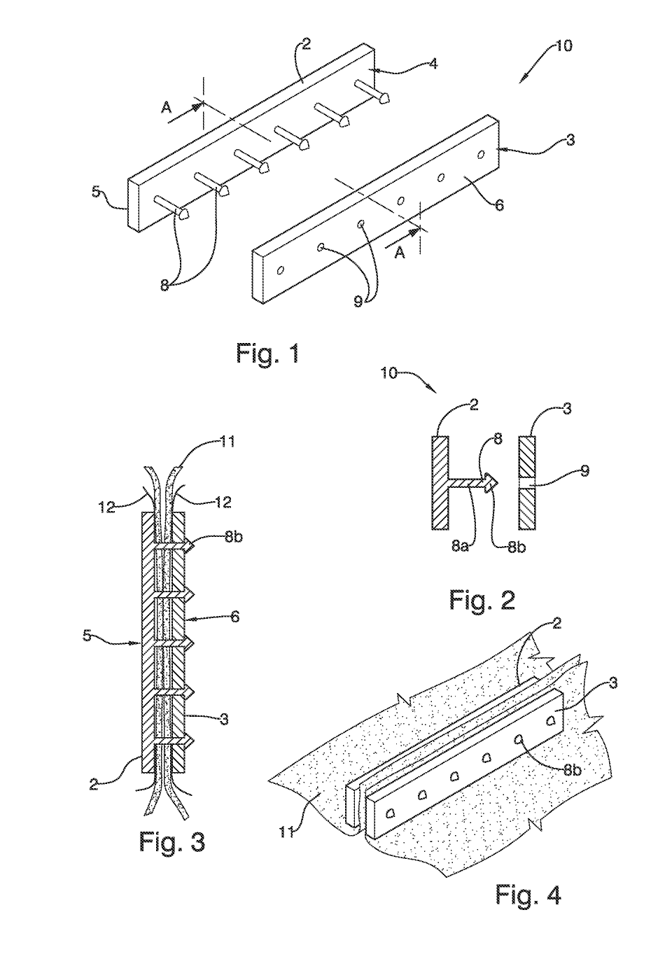

Referring now to the first preferred embodiment of the present invention, illustrated in FIG. 1, the invention is directed to a tissue approximating device 10 comprising a two-piece anastomosis clamp that can be used to approximate two tissue sections together. The clamp includes a first member 2 and a second member 3, where the clamp members are operable configured to fasten together to clamp and hold tissue, in juxtaposition to establish an anastomosis.

Said first clamp member 2 includes a front face 4 and a rear face 5. On said front face 4 a set of perpendicularly arranged and equally spaced spikes 8 are included. Each spike presents a shape similar to an arrow, with a rectilinear thing body 8a and a V-shaped or blunt rounded, or cone shaped tip 8b at its end.

Said second member 3 in turn comprises a substantially rectangular rectilinear body with a front face 7 and a rear face 6. A set of passing through equally-spaced orifices 9 are included on said faces 6-7. Said orifices have a complementary arrangement with the above cited spikes 8, so each spike 8 of the first member 2 can fit in each orifice 9 of the second member 3.

FIG. 1 illustrates the first embodiment of the purposed invention. This is an exploded perspective showing both members in a general perspective view. Both members are separated and they are in the position they will have before the clamp is fired. Even though it is not illustrated in the present set of drawings, these first and second members will be loaded in a clamp firing device during the surgical procedure, as will be explained below.

FIG. 2 shows in a cross-sectional view the first and second member in detail. Each spike 8 presents an arrow-like shape with a central linear body 8a and a V-shaped or blunt rounded, or cone shaped tip 8b. The reason behind this form is related to the purpose of this clamp device. Once the clamp is fired, the spike 8 will dig through the upper and lower tissue walls there are sandwiched in-between, into the small orifices 9. Thus, the V-shaped or blunt rounded, or cone shaped tip 8b will be retained on face 6 of the second member 3 because the internal diameter of each orifice 9 is slightly lesser than the diameter of each tip 8b. By retaining the tip 8b on face 6 of said second member 3, the loosening of said spike 8 is prevented.

FIG. 3 shows another cross sectional view, this time illustrating a piece of tissue 11 to which the purposed clamp device 10 was placed. FIG. 4 illustrates the same situation but this time through a general perspective view.

As it is shown in these figures, once the purposed approximating device 10 is placed, the tissue 11 is joined to form a proper anastomosis. The clamp 10 is fired by the surgeon, and the spikes 8 dig through the tissue 11 and passed through the orifices 9. Because of the special shape of tips 8b, said spikes 8 are locked in place on said face 6 of said second member 3 retaining at the same time both parts of the tissue 11 together. When the clamp is released the soft absorbable material will be attached around the outside of the tissue 11, with the spikes 8 securing both walls together.

It is important to point out that over each of said first and second members 2-3 a curtain-like covering 12 is placed or is already incorporated into the first and second member. Furthermore, during the procedure the surgeon will cover both lips of the tissue to be anastomosed with the same covering 12. This may be done be using two independent curtaineous coverings, or by using one curtaineous covering that can be passed through slits in each of the members to better accommodate the covering about the anastomosis. In addition one single covering may be fixed about the members and may incorporate both members (spikes and slits).

While the preferred embodiments of the invention have been described above, it will be recognized and understood that various modifications can be made in the invention and the appended claims are intended to cover all such modifications which may fall within the spirit and scope of the invention

I claim:

1. Tissue approximating device, comprising a two-piece anastomosis clamp including a first member and a second member, the first clamp member includes a set of spikes and the second clamp member includes a set of receptacles for receiving said spikes; being both members made of an absorbable material.

2. Tissue approximating device, in accordance to claim 1, wherein the first member and the second member have a rectilinear shape.

3. Tissue approximating device, in accordance to claim 1, wherein the first member and the second member have a curved shape.

4. Tissue approximating device, in accordance to claim 1, wherein each spike has an arrow-like shape, with a rectilinear thin body and a V-shape shaped tip at its end.

5. Tissue approximating device, in accordance to claim 1, wherein each spike has an arrow-like shape, with a rectilinear thin body and a blunt rounded tip at its end.

6. Tissue approximating device, in accordance to claim 1, wherein each spike has an arrow-like shape, with a rectilinear thin body and a cone-shaped tip at its end.

7. Tissue approximating device, in accordance to claim 1, wherein, a soft curtain-like covering made also of an absorbable material is placed over said first and second members and around the tissue to be anastomosed.

8. Tissue approximating device, in accordance to claim 1, wherein, a soft curtain-like covering made also of a biodegradable material is placed over said first and second members and around the tissue to be anastomosed.

9. Tissue approximating device, in accordance to claim 1, wherein the anastomosis is performed via an open technique.

10. Tissue approximating device, in accordance to claim 1, wherein the anastomosis is performed via Natural Orifice Translumenal Endoscopic Surgery (NOTES).

11. Tissue approximating device, in accordance to claim 1, wherein the anastomosis is performed via single site surgery.
